(19) United States
(12) Patent Application Publication
(10) Pub. No.: US 2010/0072400 A1
(43) Pub. Date: Mar. 25, 2010

(54) METHOD FOR CLEANING A HIGH RESOLUTION SCANNING ELECTRON MICROSCOPE SAMPLE WITH A LOW POWER ION BEAM

(75) Inventors: TIEN-YOU CHEN, Taipei County (TW); PEI-YI CHEN, Taoyuan County (TW); BI-JEN CHEN, Taipei County (TW)

Correspondence Address: ROSENBERG, KLEIN & LEE, 3458 ELLICOTT CENTER DRIVE-SUITE 101, ELLICOTT CITY, MD, 21043, US

(73) Assignee: INOTERA MEMORIES, INC., Taoyuan County (TW)

(21) Appl. No.: 12/369,999

(22) Filed: Feb. 12, 2009

(30) Foreign Application Priority Data
Sep. 24, 2008 (TW) 97136601

(51) Int. Cl. A61N 5/00

(52) U.S. Cl. 250/492.3

(57) ABSTRACT

A method for cleaning a high resolution electron microscope sample with a low power ion beam includes the following steps. One sample is transmitted to a dual beam system to perform the milling operation. The sample includes at least one cross-sectional area. The cross-sectional area includes a plurality of active areas (AA), a plurality of gates, and a plurality of gate oxides (GOx). During the milling process, re-deposition is generated, and the re-deposition covers the active area of the cross-sectional area and the gate oxide in the middle of the gate. An ion beam is applied to the cross-sectional area to remove the re-deposition. An oxide etching operation is performed to the surface of the cross-sectional area to generate surface topography. A high resolution scanning electron microscope is used to obtain an image from the cross-sectional area. The active area and the gate oxide are checked and analyzed.

### BACKGROUND OF THE INVENTION

1. Field of the Invention

The present invention relates to a method for cleaning a high resolution electron microscope sample, especially for a method for cleaning a high resolution electron microscope sample with a low power ion beam.

2. Description of Related Art

Physical failure analysis (PFA) is important for the semiconductor manufacturing process, such as the manufacture of dynamic random access memory (DRAM). When the critical dimension (CD) of the semiconductor manufacturing process is under 10 nanometer (nm), gate induced drain leakage (GIDL) generated from the drain and bit line coupling will reduce the yield rate of the semiconductor manufacturing process so that the cost of the semiconductor manufacturing process increases.

Mechanical polishing and dual beam milling are popular analysis methods used in the semiconductor foundry.

However, for mechanical polishing, it is difficult to exactly position the detected cross-sectional surface of the semiconductor product so that the detection probability is low. When the dual beam milling is used in the semiconductor manufacturing process, re-deposition is generated during the dual beam milling, covering the detected cross-sectional surface so that the dual beam milling cannot be smoothly performed. As shown in FIG. 1 and FIG. 2, during the dual beam milling, the re-deposition covers the active area (AA) and the gate oxide (GOx) 102 in the middle of the gate. Therefore, gate induced drain leakage (GIDL) generated from the drain and bit line coupling can not be analyzed. The yield rate of the semiconductor manufacturing process is decreased and the cost of the semiconductor manufacturing process is increased.

### SUMMARY OF THE INVENTION

One particular aspect of the present invention is to provide a method for cleaning a high resolution electron microscope sample with a low power ion beam to improve the yield rate of the semiconductor manufacturing process and reduce the cost of the semiconductor manufacturing process.

The method for cleaning a high resolution electron microscope sample with a low power ion beam includes the following steps. At least one sample is provided. The sample is transmitted to a dual beam system to perform the milling operation. The sample includes at least one cross-sectional area. The cross-sectional area includes a plurality of active areas (AA), a plurality of gates, and a plurality of gate oxides (GOx). During the milling process, re-deposition is generated, and part of the re-deposition covers the active area of the cross-sectional area and the gate oxide in the middle of the gate. The re-deposition is removed from the cross-sectional area by means of an ion beam. An oxide etching operation is performed to the surface of the cross-sectional area to generate surface topography. A high resolution scanning electron microscope is used to obtain an image of the cross-sectional area. The active area of the cross-sectional area and the gate oxide in the middle of the gate is checked and analyzed.

The present invention has the following characteristics. By utilizing the dual beam system to perform the milling operation to remove the re-deposition of the active area, the cross-sectional area can be exactly measured to improve the yield rate of the semiconductor manufacturing process and reduce the cost of the semiconductor manufacturing process.

For further understanding of the present invention, reference is made to the following detailed description illustrating the embodiments and examples of the present invention. The description is for illustrative purpose only and is not intended to limit the scope of the claim.

### BRIEF DESCRIPTION OF THE DRAWINGS

The drawings included herein provide a further understanding of the present invention. A brief introduction of the drawings is as follows:

FIG. 1 is a schematic diagram of the cross-sectional area along one word line viewed from a high resolution electron microscope of the prior art;

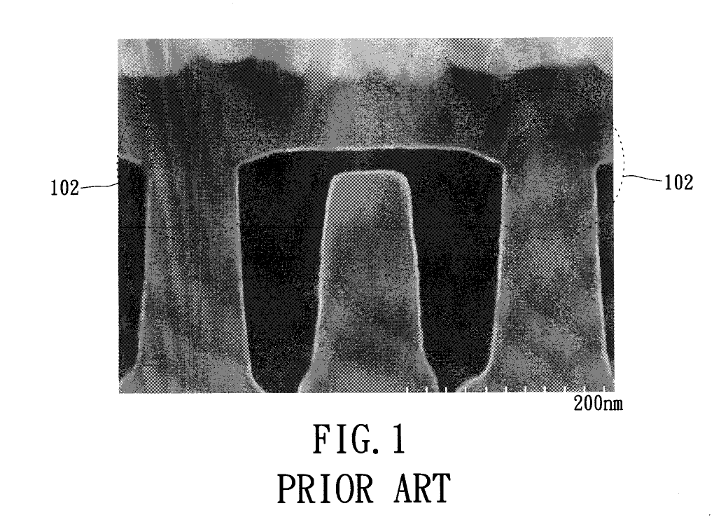

FIG. 2 is a schematic diagram of the cross-sectional area along one bit line viewed from a high resolution electron microscope of the prior art;

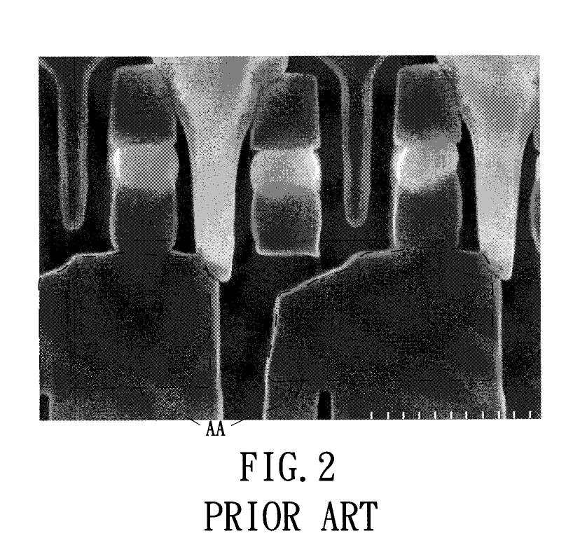

FIG. 3 is a flow chart of the method for cleaning a high resolution electron microscope sample with a low power ion beam of the present invention;

FIG. 4 is a schematic diagram of the cross-sectional area along one word line viewed from a high resolution electron microscope before the ion beam is applied;

FIG. 5 is a schematic diagram of the cross-sectional area along one bit line viewed from a high resolution electron microscope before the ion beam is applied;

FIG. 6 is a schematic diagram of the cross-sectional area along one word line viewed from a high resolution electron microscope after the ion beam is applied; and

FIG. 7 is a schematic diagram of the cross-sectional area along one bit line viewed from a high resolution electron microscope after the ion beam is applied.

### DETAILED DESCRIPTION OF PREFERRED EMBODIMENTS

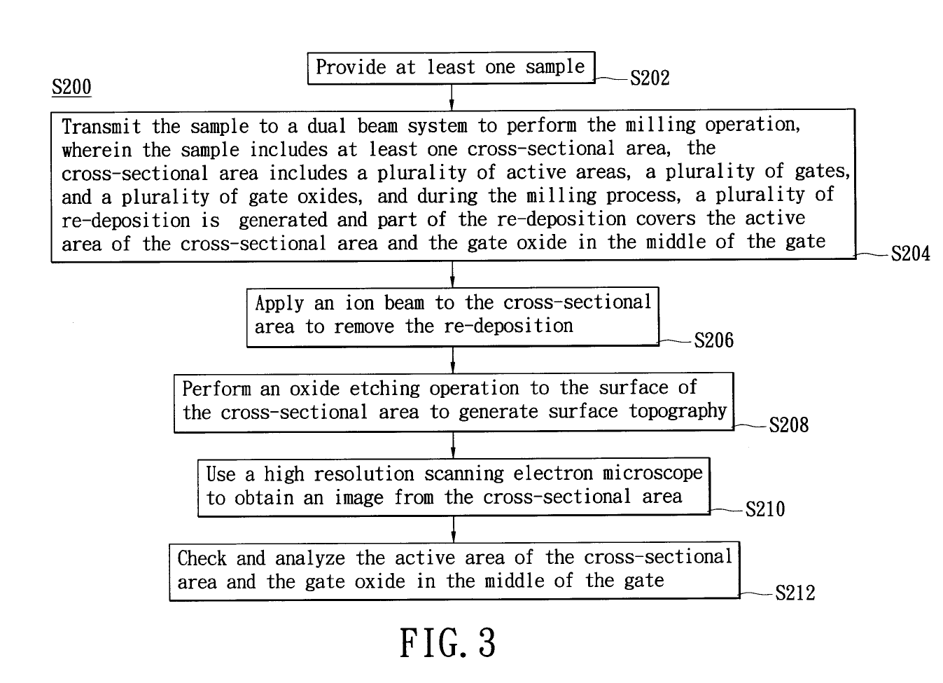

Reference is made to FIG. 3. The method for cleaning a high resolution electron microscope sample with a low power ion beam S200 includes the following steps.

Step S202 is performed. At least one sample is provided. In this embodiment, the sample is a semiconductor product.

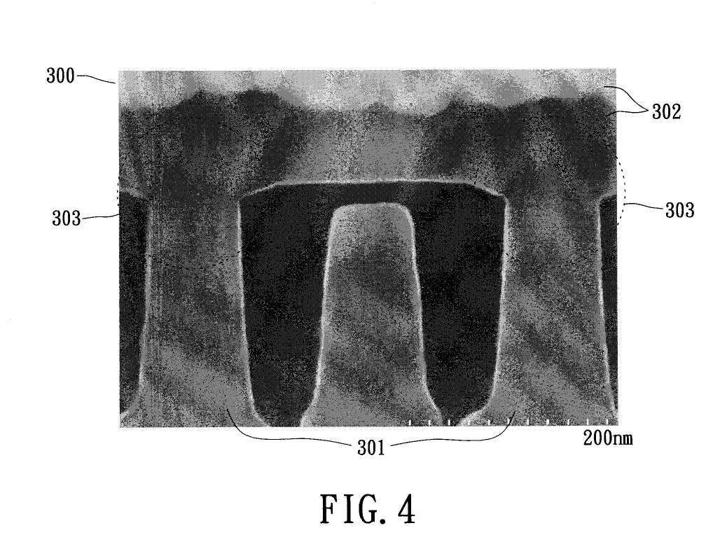

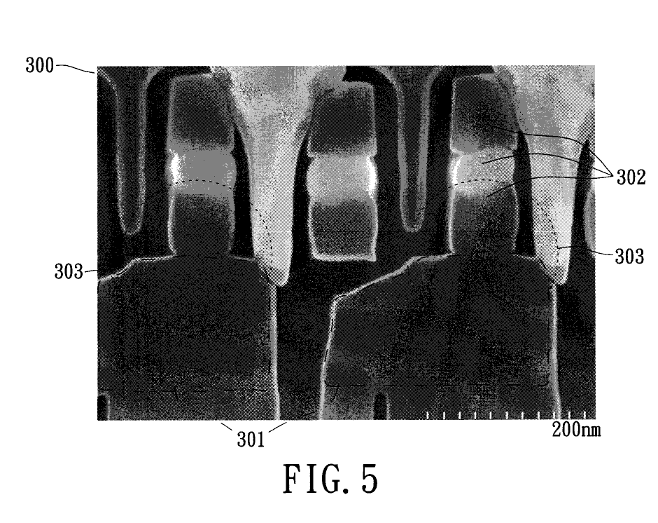

Step S204 is performed. The sample is transmitted to a dual beam system to perform the milling operation. As shown in FIGS. 4 and 5, which show the schematic diagram of the cross-sectional area along one word line and one bit line of the sample viewed from a high resolution electron microscope. The sample includes at least one cross-sectional area 300. The cross-sectional area 300 includes a plurality of active areas (AA) 301, a plurality of gates 302, and a plurality of gate oxides (GOx) 303. During the milling process, re-deposition, composed mainly of Si, cover the active area 301 of the cross-sectional area 300 and the gate oxide 303 in the middle of the gate 302.

The dual beam system also is called as focused ion beam (FIB) system, and is a microscope milling equipment that uses an electric lens to focus the ion beam into a tiny area. The ion beam is a liquid metal ion source (LMIS). The metal material is Gallium (Ga) due to the Ga element has a low melting point, a low vapor pressure, and a good oxidization-resistance. The dual beam system includes liquid metal ion source, electric lens, scanning electrode, sub-particle detector, sample base with five to seven axis moving, vacuum system, anti-vibration and anti-magnetic filed device, electronic control panel, and computer. The operation principle is to exert the suppressor to the liquid metal ion source to make the liquid Ga form a tiny tip. Next, an extractor is exerted to guide the tiny tip Ga to form the ion beam. Under a general operation voltage, the current density of the tiny tip is 100 10⁻¹⁰ AMP/CM₂. By using an electric lens to focus and passing through the automatic variable aperture (AVA) to determine the size of the ion beam, and focusing onto the sample surface second times, the physical pumping algorithm are utilized to achieve the milling process. In this embodiment, the voltage for the dual beam system to perform the milling operation is 30 kilovolt (kV), and the current is 48 picoampere (pA). The sample is transmitted to the dual beam system. The angle between the sample and the machine table surface is 52 degrees. In other words, the angle between the current of the dual beam system and the machine table surface is 52 degrees. The dimension of the contact area of the cross-sectional area 300 for applying the ion beam is 10 micro-meters (μm)×2 (μm). The shape of the active areas is recessed.

Step S206 is performed. An ion beam is applied to the cross-sectional area 300 to remove the re-deposition. In this embodiment, the voltage for generating the ion beam is about 10 kV. The angle between the cross-sectional area 300 and the machine table surface is about 50 degrees.

Step S208 is performed. An oxide etching operation is performed to the surface of the cross-sectional area 300 to generate surface topography. Thereby, the electronic signal of the image for a high resolution scanning electron microscope is enhanced.

Step S210 is performed. A high resolution scanning electron microscope is used to obtain an image from the cross-sectional area 300.

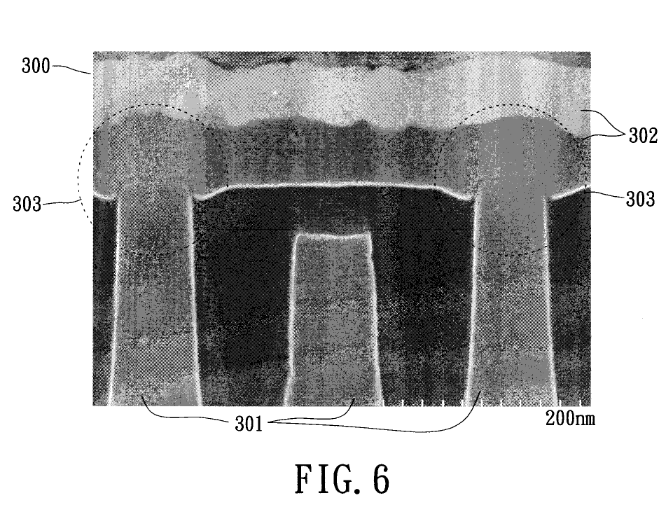

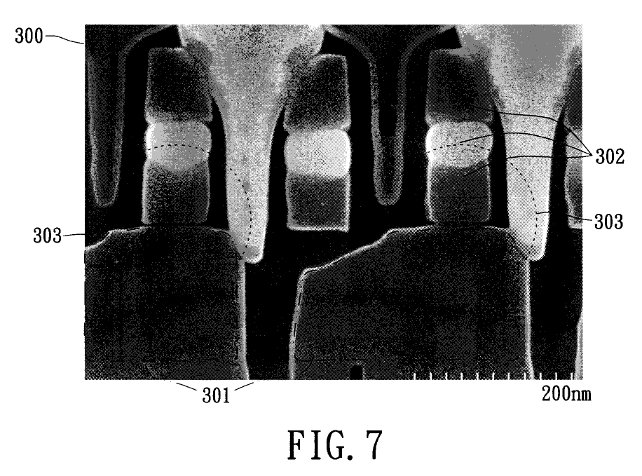

Step S212 is performed. The active area 301 of the cross-sectional area 300 and the gate oxide 303 in the middle of the gate 302 is checked and analyzed, referring to FIGS. 6 and 7. In this embodiment, the check and analysis process further includes measuring the parameters of the gate oxide 303. The parameters of the gate oxide 303 include the width of the gates 302 and the depth of the gates 302.

The present invention has the following characteristics. By utilizing the dual beam system to perform the milling operation to remove the re-deposition of the active area, the cross-sectional area can be exactly measured to improve the yield rate of the semiconductor manufacturing process and reduce the cost of the semiconductor manufacturing process.

The description above only illustrates specific embodiments and examples of the present invention. The present invention should therefore cover various modifications and variations made to the herein-described structure and operations of the present invention, provided they fall within the scope of the present invention as defined in the following appended claims.

What is claimed is:

1. A method for cleaning a high resolution electron microscope sample with a low power ion beam, comprising:
providing at least one sample;
transmitting the sample to a dual beam system to perform a milling operation, wherein the sample includes at least one cross-sectional area, the cross-sectional area includes a plurality of active areas, a plurality of gates, and a plurality of gate oxides, and during the milling process, re-deposition is generated, part of which cover the active area of the cross-sectional area and the gate oxide in the middle of the gate;
applying an ion beam to the cross-sectional area to remove the re-deposition;
performing an oxide etching operation to the surface of the cross-sectional area to generate surface topography;
using a high resolution scanning electron microscope to obtain an image from the cross-sectional area; and
checking and analyzing the active area of the cross-sectional area and the gate oxide in the middle of the gate.

2. The method for cleaning a high resolution electron microscope sample with a low power ion beam as in claim 1, wherein the step of checking and analyzing the active area of the cross-sectional area and the gate oxide in the middle of the gate further comprises measuring the parameters of the gate oxide, the parameters of the gate oxide include the width of the gates and the depth of the gates, and the sample is a semiconductor product.

3. The method for cleaning a high resolution electron microscope sample with a low power ion beam as in claim 2, wherein the voltage for the dual beam system to perform the milling operation is 30 kV.

4. The method for cleaning a high resolution electron microscope sample with a low power ion beam as in claim 2, wherein the current for the dual beam system to perform the milling operation is 48 pA.

5. The method for cleaning a high resolution electron microscope sample with a low power ion beam as in claim 2, wherein the angle between the sample and the machine table surface is 52 degrees in the step of transmitting the sample to a dual beam system.

6. The method for cleaning a high resolution electron microscope sample with a low power ion beam as in claim 2, wherein the dimension of the contact area of the cross-sectional area for applying the ion beam is 10 μm×2 μm.

7. The method for cleaning a high resolution electron microscope sample with a low power ion beam as in claim 2, wherein the main component of the re-deposition is Si.

8. The method for cleaning a high resolution electron microscope sample with a low power ion beam as in claim 2, wherein the voltage for generating the ion beam is 10 kV.

9. The method for cleaning a high resolution electron microscope sample with a low power ion beam as in claim 2, wherein the shape of the active area is recessed.

10. The method for cleaning a high resolution electron microscope sample with a low power ion beam as in claim 2, wherein the angle between the cross-sectional area and the machine table surface is about 50 degrees in the step of applying an ion beam to the cross-sectional area.

11. A method for cleaning a high resolution electron microscope sample with a low power ion beam, comprising:
providing at least one sample;
transmitting the sample to a dual beam system to perform the milling operation, wherein the sample includes at least one cross-sectional area, and during the milling process, re-deposition is generated which cover the cross-sectional area;
applying an ion beam to the cross-sectional area to remove the re-deposition;
performing an oxide etching operation to the surface of the cross-sectional area to generate surface topography; and
using a high resolution scanning electron microscope to obtain an image from the cross-sectional area, and checking and analyzing the cross-sectional area.

12. The method for cleaning a high resolution electron microscope sample with a low power ion beam as in claim 11, wherein the sample is a semiconductor product.

13. The method for cleaning a high resolution electron microscope sample with a low power ion beam as in claim 11, wherein the voltage for the dual beam system to perform the milling operation is 30 kV.

14. The method for cleaning a high resolution electron microscope sample with a low power ion beam as in claim 11, wherein the current for the dual beam system to perform the milling operation is 48 pA.

15. The method for cleaning a high resolution electron microscope sample with a low power ion beam as in claim 11, wherein the angle between the sample and the machine table surface is 52 degrees in the step of transmitting the sample to a dual beam system.

16. The method for cleaning a high resolution electron microscope sample with a low power ion beam as in claim 11, wherein the dimension of the contact area of the cross-sectional area for applying the ion beam is 10 μm×2 μm.

17. The method for cleaning a high resolution electron microscope sample with a low power ion beam as in claim 11, wherein the main component of the re-deposition is Si.

18. The method for cleaning a high resolution electron microscope sample with a low power ion beam as in claim 11, wherein the voltage for generating the ion beam is 10 kV.

19. The method for cleaning a high resolution electron microscope sample with a low power ion beam as in claim 11, wherein the shape of the active area is divot.

20. The method for cleaning a high resolution electron microscope sample with a low power ion beam as in claim 11, wherein the angle between the cross-sectional area and the machine table surface is about 50 degrees in the step of applying the ion beam to the cross-sectional area.
